(19) United States
(12) Patent Application Publication
(10) Pub. No.: US 2007/0219443 A1
(43) Pub. Date: Sep. 20, 2007

(54) MAGNETIC RESONANCE MARKER BASED POSITION AND ORIENTATION PROBE

(75) Inventors: Gosta Ehnholm, Helsinki (FI); Matti Lindstrom, Helsinki (FI)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 11/574,520

(22) PCT Filed: Aug. 25, 2005
(86) PCT No.: PCT/IB05/52792

§ 371 Date: Mar. 1, 2007

Related U.S. Application Data
(60) Provisional application No. 60/606,258, filed on Sep. 1, 2004.

(51) Int. Cl. A61B 5/055; G01R 33/28

(52) U.S. Cl. 600/414

(57) ABSTRACT

A magnetic resonance position and orientation marking system includes fiducial assembly (30) with at least three fiducial markers (31, 32, 33) each coupled with at least one magnetic resonance receive coil (70, 74, 80, 84). At least one of the fiducial markers has at least one of: (i) marker nuclei selectively excitable over ¹H fat and water resonance, 5 and (ii) a plurality of magnetic resonance receive coils (70, 84) coupled therewith. At least two magnetic resonance receive channels (40, 42) receive magnetic resonance signals from the at least three fiducial markers (31, 32, 33) responsive to excitation of magnetic resonance in said at least three fiducial markers by a magnetic resonance imaging scanner (10).

The following relates to the magnetic resonance arts. It finds particular application in interventional magnetic resonance imaging in which magnetic resonance imaging is used to monitor a biopsy or other interventional medical procedure, and will be described with particular reference thereto. However, it also finds application in magnetic resonance imaging generally.

In interventional medical procedures such as biopsies, thermal ablations, brachytherapy, and so forth, it is important to have precise knowledge of the position and orientation of the biopsy needle, catheter, or other interventional instrument as the interventional procedure progresses. In non-interventional procedures, position and orientation tracking can also be useful, for example as a tool for slice selection based on anatomical landmarks. In some approaches, a magnetic resonance imaging scanner is used to image the patient during the interventional medical procedure and another, non-magnetic resonance-based, technique is used to track the position and orientation of the interventional instrument. For example, Philips Optoguide™ employs a stereoscopic camera pair that monitors optical markers to determine the position and orientation of the interventional instrument. In this approach, the optical markers must remain within the line-of-sight of the monitoring cameras during the tracking. Moreover, the optical monitoring system must be spatially calibrated with respect to the magnetic resonance imaging.

Magnetic resonance imaging has also been used to simultaneously provide both images of the patient and information for tracking the interventional instrument. In some approaches, the magnetic resonance-based tracking takes advantage of susceptibility artifacts superimposed upon the magnetic resonance image by the tip of the interventional instrument. This approach has the disadvantage of disturbing the image of the region around the instrument tip, and also typically does not provide enough information to extract both spatial and angular information.

In other approaches, a dedicated fiducial assembly is provided in a fixed, known spatial relationship respective to the interventional instrument. In these approaches, the fiducial assembly includes at least three spatially separated magnetic fiducial markers, each producing a separate magnetic resonance signal. Three magnetic resonance receive channels independently acquire and process magnetic resonance from the three magnetic markers in parallel, which requires a threefold duplication of hardware. Moreover, the ¹H proton magnetic resonance signal emanating from the patient can interfere with the magnetic resonance marking and tracking.

The present invention contemplates improved apparatuses and methods that overcome the aforementioned limitations and others.

According to one aspect, a magnetic resonance position and orientation marking system is disclosed. A fiducial assembly includes at least three fiducial markers each coupled with at least one magnetic resonance receive coil. At least one of the fiducial markers has at least one of: (i) marker nuclei selectively excitable over ¹H fat and water resonance, and (ii) a plurality of magnetic resonance receive coils. At least two magnetic resonance receive channels receive magnetic resonance signals from the at least three fiducial markers responsive to excitation of magnetic resonance in said at least three fiducial markers by an associated magnetic resonance imaging scanner.

According to another aspect, a method is provided for determining position and orientation of a fiducial assembly including at least three fiducial markers. Magnetic resonance is excited in the at least three fiducial markers. Each fiducial marker is coupled with at least one magnetic resonance receive coil. At least one of the fiducial markers has at least one of: (i) marker nuclei selectively excitable over ¹H fat and water resonance, and (ii) a plurality of magnetic resonance receive coils. Magnetic resonance signals are received from the excited at least three fiducial markers via at least two magnetic resonance receive channels.

One advantage resides in providing a robust magnetic resonance-based marking and tracking system of reduced cost and complexity.

Another advantage resides in providing magnetic resonance-based marking and tracking employing only two magnetic resonance receive channels.

Yet another advantage resides in providing a magnetic resonance-based marking and tracking system in which interference from ¹H resonance emanating from the imaging subject is substantially reduced.

Still another advantage resides in providing robust and reliable resolution of marking and tracking ambiguities arising from fiducial marker overlaps, symmetric marker configurations, and the like.

Numerous additional advantages and benefits will become apparent to those of ordinary skill in the art upon reading the following detailed description of the preferred embodiments.

The invention may take form in various components and arrangements of components, and in various process operations and arrangements of process operations. The drawings are only for the purpose of illustrating preferred embodiments and are not to be construed as limiting the invention.

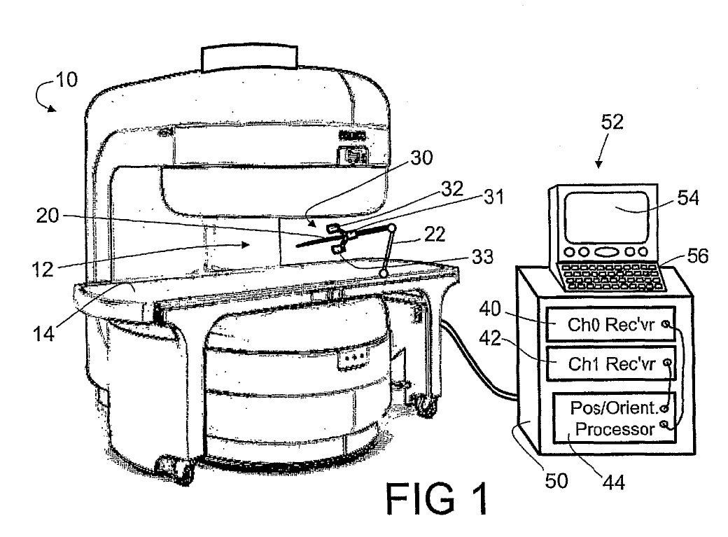

FIG. 1 shows an interventional magnetic resonance system including an example interventional instrument and a tracking system for tracking the interventional instrument.

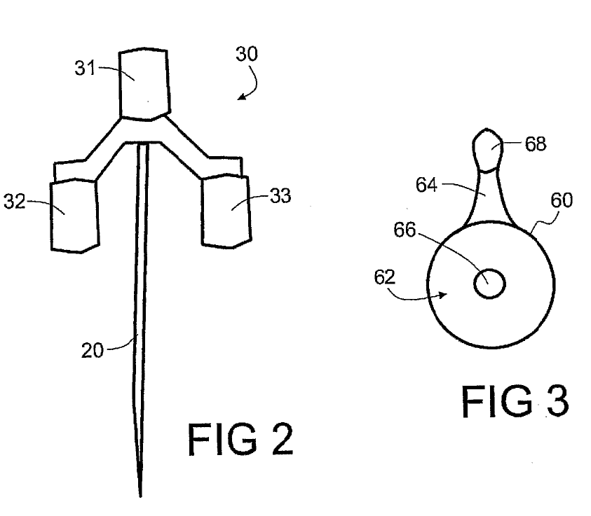

FIG. 2 shows the interventional instrument of FIG. 1 with the fiducial assembly secured therewith.

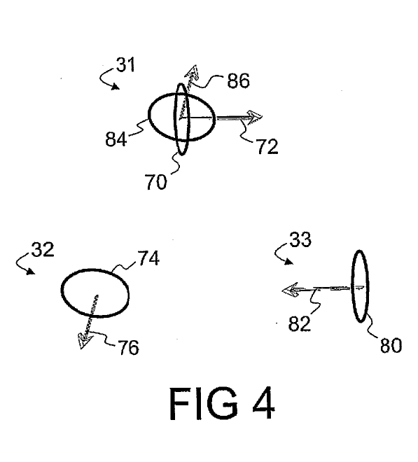

FIG. 3 shows a vial of magnetic marker material which is suitable for use as one of the fiducial markers of the fiducial assembly of FIG. 2.

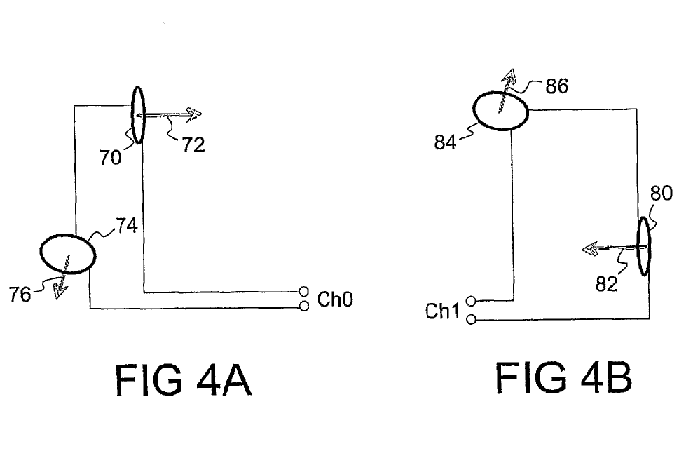

FIG. 4 diagrammatically shows the coil orientations of the receive coils of the fiducial assembly of FIG. 2.

FIG. 4A diagrammatically shows the electrical layout of the “Ch0” receive channel of the fiducial assembly of FIG. 2.

FIG. 4B diagrammatically shows the electrical layout of the “Ch1” receive channel of the fiducial assembly of FIG. 2.

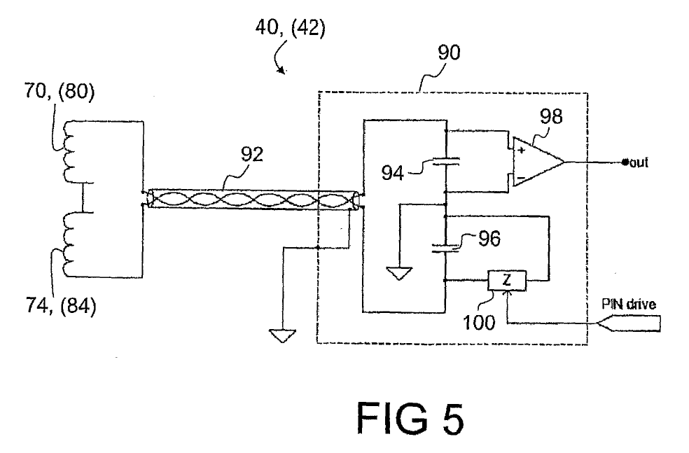

FIG. 5 shows a simplified example electrical schematic of a pre-amplifier suitable for use in the magnetic resonance channel receivers of the system of FIG. 1.

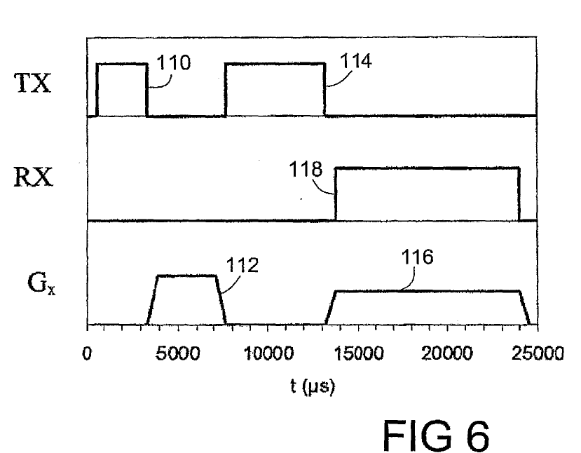

FIG. 6 diagrammatically shows a suitable magnetic resonance pulse sequence for measuring a one-dimensional projection along the x-direction.

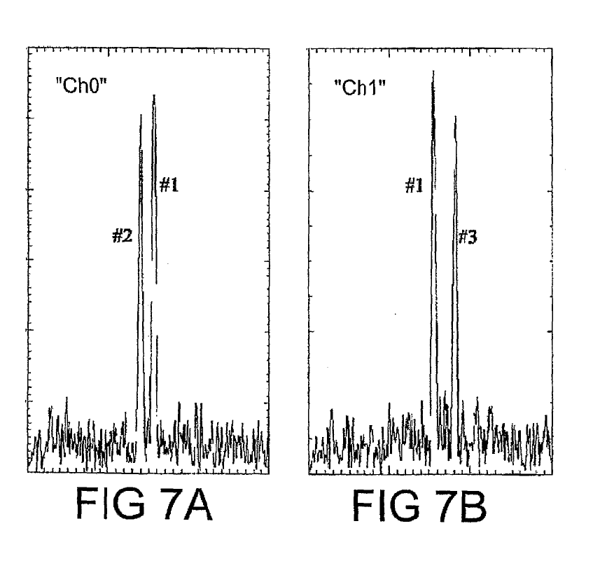

FIGS. 7A and 7B show Fourier-transformed frequency domain spectra measured for “Ch0” and “Ch1”, respectively, for a selected one-dimensional projection.

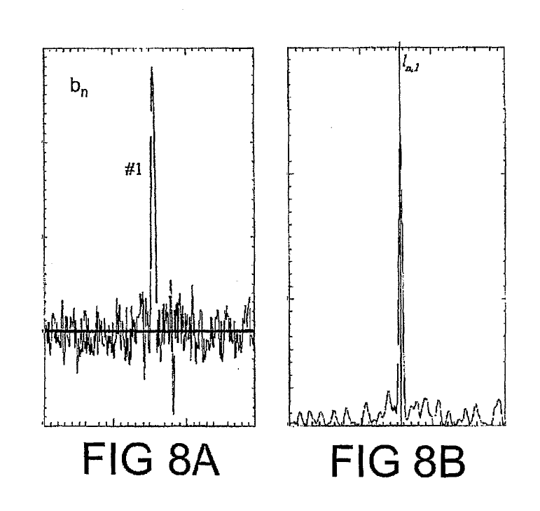

FIG. 8A shows a multiplicative combination of the “Ch0” and “Ch1” spectra of FIGS. 7A and 7B.

FIG. 8B shows the multiplicative combination of FIG. 8A after smoothing and Fourier interpolation.

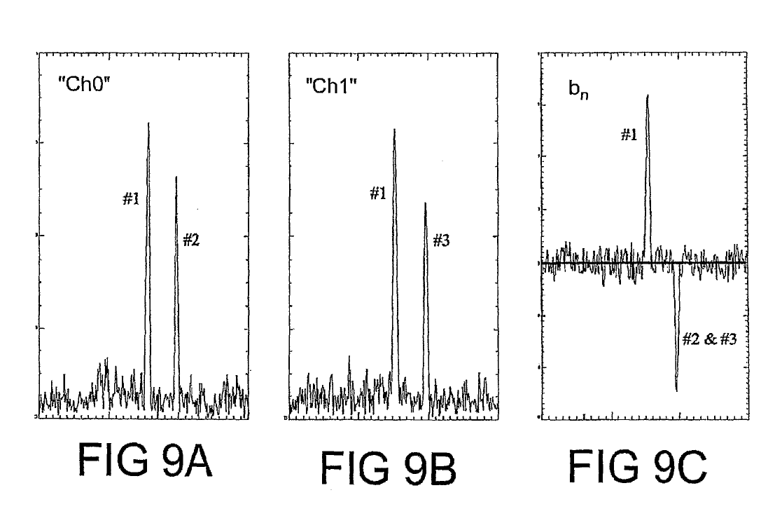

FIGS. 9A and 9B show Fourier-transformed frequency domain spectra measured for “Ch0” and “Ch1”, respectively, for a selected one-dimensional projection in which two fiducial marker peaks strongly overlap.

FIG. 9C shows a multiplicative combination of the “Ch0” and “Ch1” spectra of FIGS. 9A and 9B. The overlapping peaks correspond to a negative peak in the multiplicative combination of FIG. 9C.

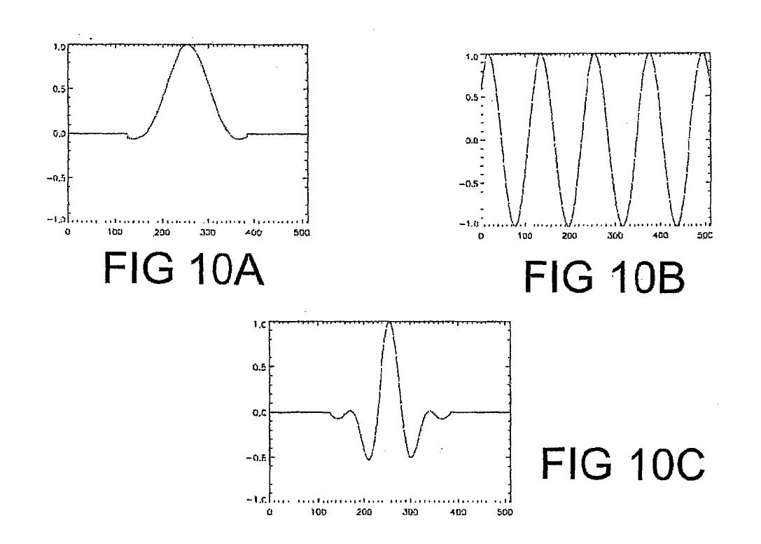

FIGS. 10A, 10B, and 10C illustrate construction of a shifted time domain shape approximating the shape of the first fiducial marker in the “Ch0” and “Ch1” channel data.

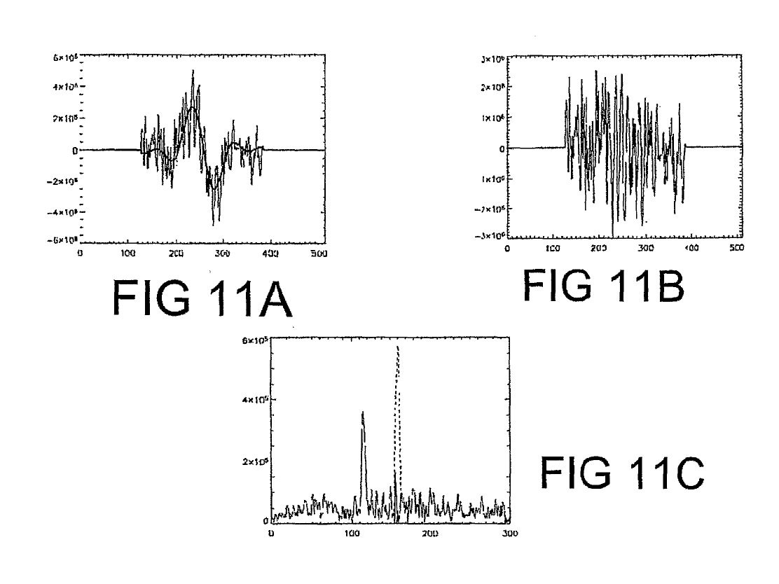

FIGS. 11A, 11B, and 11C illustrate identification of the “#2” peak due to the second fiducial marker in the “Ch0” data using the shifted time domain shape of FIG. 10C.

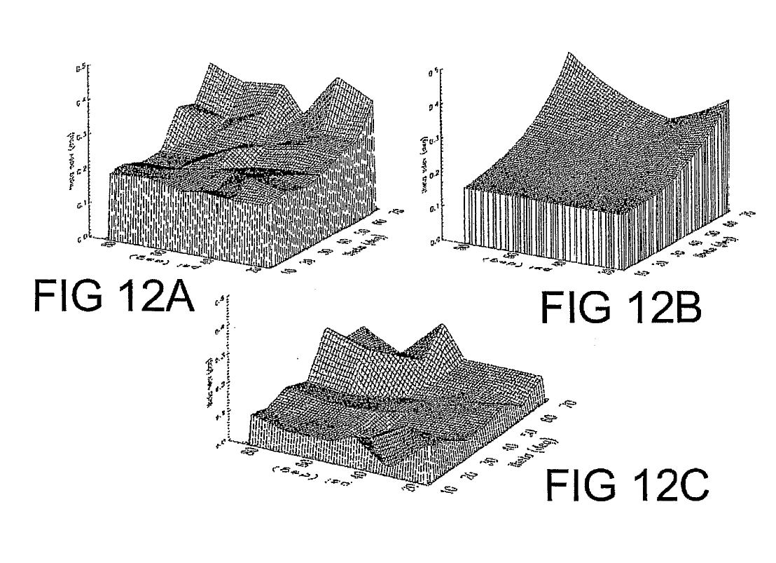

FIG. 12A shows the measured standard deviation of θ, where the data is regridded, measurement points lie at θ=10°, 20°, . . . 70°, and ψ=15°, 30°, 45°, 52°, 60°, 67°, 75°, and 82°.

FIG. 12B shows theoretical predictions of the standard deviation assuming linear dependence between inverse signal-to-noise ratios of the derived channels representing each peak “#1”, “#2”, “#3” and statistical angle fluctuation.

FIG. 12C shows the measured rotation-dependent errors of θ.

With reference to FIG. 1, a magnetic resonance imaging scanner 10 performs magnetic resonance imaging in a region of interest 12. In the illustrated embodiment, the magnetic resonance imaging scanner 10 is a Philips Panorama 0.23T scanner available from Philips Medical Systems Nederland B.V. This scanner has an open bore that facilitates interventional medical procedures. It will be appreciated that the scanner 10 is an example only, and that the instrument marking and tracking methods and apparatuses described herein are generally applicable in conjunction with substantially any type of magnetic resonance imaging scanner, including but not limited to open bore scanners, closed-bore scanners, vertical bore scanners, and so forth. An imaging subject (not shown), such as a human medical patient, is placed on a subject support 14 and positioned within the region of interest 12 of the scanner 10.

In an interventional medical procedure, an interventional instrument 20, such as a biopsy needle, a catheter, pointer, or the like, is employed to perform a biopsy, a thermal ablation treatment, brachytherapy, slice selection, or so forth. The magnetic resonance imaging scanner 10 images the area of the procedure and the interventional instrument 20 during the interventional medical procedure to provide visual guidance to the surgeon or other medical therapist. In some interventional procedures, the interventional instrument is manipulated directly by the surgeon or other medical therapist. However, for delicate or sensitive procedures which call for high precision manipulation of the interventional instrument 20, a mechanical assembly 22 supports and manipulates the interventional instrument 20, or aids in the positioning of the interventional instrument 20, under the direction of the surgeon or other medical therapist. In the illustrated embodiment, the mechanical assembly 22 is mounted to the subject support 14; however, in other contemplated embodiments the arm may be supported or mounted on the scanner 10 or on another associated structure.

Regardless of how the interventional instrument 20 is manipulated, it is advantageous to provide automated marking and tracking of the instrument 20 during the interventional procedure. Toward this end, a fiducial assembly 30 is disposed on the interventional instrument 20 within the field of view of the magnetic resonance imaging scanner 10. The fiducial assembly 30 includes, in the illustrated embodiment, three fiducial markers 31, 32, 33 that produce magnetic resonance signals responsive to a radio frequency excitation generated by the magnetic resonance imaging scanner 10. Three markers is generally sufficient to determine the spatial position and orientation of the interventional instrument 20; however, additional markers can be included to provide redundancy and improved tracking robustness. In the illustrated embodiment, the three fiducial markers 31, 32, 33 are monitored by two radio frequency channel receivers 40, 42 that produce two quadrature magnetic resonance receive signals designated herein as “Ch0” and “Ch1”, respectively. These two magnetic resonance receive signals are processed by a position/orientation processor 44 to determine the position and orientation of the fiducial assembly 30, and thus the position and orientation of the interventional instrument 20 that is rigidly connected with the fiducial assembly 30. Alternatively, each fiducial marker 31, 32, 33 can be monitored by a separate magnetic receiver channel (that is, three receiver channels in all) and the three channels received and suitably processed to determine position and orientation.

In the illustrated embodiment, the two radio frequency channel receivers 40, 42 and the position/orientation processor 44 are mounted in an electronics rack 50, and a computer 52 with a display 54 and a graphical user interface 56 serves as a user interface for the surgeon or other medical therapist to receive position and orientation information pertaining to the interventional instrument 20. In the illustrated embodiment, the computer 52 also provides a user interface for control of the magnetic resonance imaging scanner 10 and for receiving images therefrom. It is to be appreciated that this hardware configuration is an illustrative example only, which those skilled in the art can readily modify. For example, the position/orientation processor 44 can be embodied by computational software executed by the computer 52, rather than as a separate electronics component. The two radio frequency channel receivers 40, 42 can similarly be integrated into the computer 52, for example as optional electronics cards with edge connectors that mate with the computer motherboard. In other example modifications, the computer for controlling the scanner 10 and for displaying images therefrom can be separate and distinct from the hardware used for marking and tracking the interventional instrument 20.

With continuing reference to FIG. 1 and with further reference to FIG. 2, the fiducial assembly 30 includes the three fiducial markers 31, 32, 33 which in the illustrated embodiment are positioned at the corners of an equilateral triangle, although other non-linear arrangements are contemplated. The fiducial assembly 30 is rigidly attached with the interventional instrument 20 providing a priori knowledge of the position and orientation of the fiducial assembly 30 relative to the position and orientation of the interventional instrument 20 and the location of its tip.

With reference to FIG. 3, each of the three fiducial markers 31, 32, 33 includes a sealed vial 60 containing a magnetic marker material 62. In some embodiments, the magnetic marker material 62 is a fluorine-containing material. One suitable fluorine-containing magnetic marker material is a trifluoroacetic acid solution consisting of 89 wt % trifluoroacetic acid (CAS no. 76-05-1) and 1 wt % water. Optionally, a suitable T₂ relaxation time shortening agent is added to shorten the T₂ relaxation time from over 120 milliseconds to about 25 milliseconds. For example, the T₂ relaxation time shortening agent can be manganese dichloride (MnCl₂) added to the trifluoroacetic acid solution to a final concentration of 7 millimoles-per-liter. The vials 60 should be small so as to limit interference with manipulation of the interventional instrument 20, but should also be large enough to contain enough magnetic marker material 62 to provide an adequate magnetic resonance signal. In the illustrated embodiment, the vials 60 are substantially spherical, with about a 10 millimeter outer diameter and about a 9.5 millimeter inner diameter. In the illustrated embodiment, the vials 60 are sealed by melting a neck region 64, which leaves a blob of melted glass 68 and an air bubble 66. The illustrated fiducial marker is an example—those skilled in the art can use other liquid or solid magnetic marker materials containing fluorine, hydrogen, or other nuclei suitable for generation of a magnetic resonance marking signal, and can use other suitable containers or fixtures for the magnetic marker material.

With continuing reference to FIG. 3, the vials 60 are placed inside plastic coil holders and secured by epoxy casting. The coil holders are shaped to accept a suitable magnetic resonance receive coil. This arrangement advantageously places the coil in close proximity with the magnetic marker material 62 to provide strong electromagnetic coupling therebetween. However, other coil arrangements which provide adequate electromagnetic coupling with the magnetic marker material can be used.

With continuing reference to FIGS. 1-3 and with further reference to FIGS. 4, 4A, and 4B, the first fiducial marker 31 includes a coil 70 having a coil normal 72 oriented in a first direction. The second fiducial marker 32 includes a coil 74 having a coil normal 76 oriented in a second direction different from the first direction. In the illustrated embodiment, the coil normals 72, 76 are mutually orthogonal. As shown in FIG. 4A, the two coils 70, 74 are connected in series to define the “Ch0” signal that is received by the “Ch0” receiver 40 shown in FIG. 1. (For illustrative clarity, the coils and electrical interconnections are illustrated diagrammatically in FIGS. 4, 4A, and 4B and are omitted in FIG. 2).

The third fiducial marker 33 includes a coil 80 oriented in the same plane as the coil 70 of the first fiducial marker 31; however, the coil 80 has a coil normal 82 oriented opposite the coil normal 72 of the coil 70. That is, the coil 80 of the third fiducial marker 33 has the same spatial orientation as the coil 70 of the first fiducial marker 31, but is wound and connected with an opposite polarity. Similarly, the first fiducial marker 31 includes a second coil 84 oriented in the same plane as the coil 74 of the second fiducial marker 32; however, the coil 84 has a coil normal 86 oriented opposite the coil normal 76 of the coil 74. That is, the second coil 84 of the first fiducial marker 31 has the same spatial orientation as the coil 74 of the second fiducial marker 32, but is wound with an opposite polarity. As shown in FIG. 4B, the two coils 80, 84 are connected in series to define the “Ch1” signal that is received by the “Ch1” receiver 42 shown in FIG. 1.

With reference to FIG. 5, in one suitable embodiment, the magnetic resonance channel receivers 40, 42 each include a pre-amplifier circuit 90 connected with the series interconnected coils (that is, coils 70, 74 for the first receiver 40, and coils 80, 84 for the second receiver 42) by a twisted-pair cable 92. The pre-amplifier circuit 90 includes resonant capacitances 94, 96 and an output amplifier 98. During excitation of magnetic resonance for imaging, it is typically advantageous to detune the pre-amplifier circuit 90 to avoid overloading the circuit. Accordingly, a PIN diode actuated transmit decoupling circuit (represented by a generalized impedance 100) approximates an open circuit in receive mode, and forms a parallel resonant circuit with the lower capacitance 96 in transmit mode. It will be appreciated that the pre-amplifier circuit 90 is an illustrative example—those skilled in the art can readily modify the circuit 90 or design and build other suitable receive circuitry.

With reference to FIG. 6, the position and orientation of the fiducial assembly 30 (and thus, equivalently, the position and orientation of the interventional instrument 20) is monitored periodically, for example ten times per second, by applying a series of one-dimensional projection excitations, optionally interleaved among a selected imaging sequence, and determining the positions of the fiducial markers 31, 32, 33 from the resonance detected on the “Ch0” and “Ch1” receive channels 40, 42 responsive to these projection excitations. FIG. 6 diagrammatically shows a suitable pulse sequence for such a projection measurement. A spatially non-selective excitation pulse 110, which can be a 90° pulse or other flip angle pulse, generates magnetic resonance in matter within the region of interest 12, including in the magnetic marker material 62. A dephasing gradient pulse is applied in a selected projection direction. In the illustrated example, the dephasing gradient pulse 112 is a G, gradient pulse for producing a gradient in the x-direction. While the single Gₓ gradient pulse 112 is illustrated for simplicity, it will be appreciated that by selectively combining Gₓ, G_{y}, and G_{z} gradients projections can be produced in any arbitrary direction. A non-selective 180° pulse 114 is applied, followed by application of a read gradient (a Gₓ gradient 116 in the example x-direction projection). A readout sampling period 118 executes during the read gradient 116. In one example, 512 samples are acquired at 50 kHz with a field of view of 600 mm; however, other sampling parameters can be used. A spoiler gradient can optionally be applied after the readout, but in the illustrated embodiment the spoiler gradient is omitted due to the varying read direction used in acquiring a plurality of projections of different directions. The pulse sequence shown in FIG. 6 is an example only—those skilled in the art can readily construct other suitable pulse sequences for measuring one-dimensional projections in selected projection directions.

In some preferred embodiments in which the magnetic marker material 62 contains fluorine nuclei, the magnetic resonance channel receivers 40, 42 monitor the ¹⁹F fluorine magnetic resonance. The ¹⁹F magnetic resonance peak is about 6% lower in frequency than the ¹H hydrogen magnetic resonance peak. Since the human patient or other imaging subject is generally imaged using the ¹H resonance, the scanner 10 is typically tuned to the ¹H magnetic resonance frequency. However, even when tuned to the ¹H frequency, the radio frequency transmit components of the magnetic resonance scanner 10 may generate enough strength at the ¹⁹F resonance frequency to enable fluorine-based magnetic resonance marking. For example, in one commercial magnetic resonance imaging scanner, excitation at the ¹H magnetic resonance frequency generates about 11% of the maximum (that is, ¹H frequency) B, field at the ¹⁹F fluorine resonance frequency. This excitation strength at the ¹⁹F frequency is generally adequate to enable the coils 70, 74, 80, 84, which are closely placed relative to the magnetic marker material 62 contained in the vials 60, to detect the ¹⁹F magnetic resonances excited in the fiducial markers 31, 32, 33. In the illustrated embodiment, the receive chain of the example Panorama 0.23T scanner 10 is wideband beyond the pre-amplifier 90, and the mixer IF's are adjustable for detection and sampling purposes. Hence, the output of the preamplifier circuit 90 is advantageously processed using the same scanner receive chain as is used for proton imaging.

When using the ¹⁹F magnetic resonance, the diminished radio frequency transmit strength at the ¹⁹F frequency (as compared with the imaging ¹H frequency) calls for using comparatively longer transmit pulses, such as 2.75 milliseconds for the excitation pulse 110 and 5.50 milliseconds for the 180° pulse 114. This results in a relatively long echo time (about 17 milliseconds for the illustrated embodiment) and a correspondingly narrowband excitation, which strongly confines the fiducial marker signals to the homogeneous volume of the magnet of the scanner 10.

The ¹⁹F resonance of the example fluorine-based marker material 62 has been found to work well at B₀=0.23 Tesla. In some tracking sequences performed at 0.23 Tesla, the ¹⁹F fluorine resonance is selectively excited without substantial excitation of the ¹H water and fat resonances of the patient, which facilitates distinguishing the marker resonances from imaging subject resonances. Moreover, the ¹⁹F resonances in the three fiducial markers 31, 32, 33 are excited in the same way and precess at the same phase, which facilitates distinguishing the markers based on phase differences produced by different coil winding directions.

The ¹⁹F resonance is an example; in other embodiments other nuclear magnetic resonances are employed in the fiducial markers. In some embodiments a marker material having a ¹H resonance with a strong chemical shift of the resonance frequency is sufficient to enable selective excitation of resonance in the marker material without substantial excitation of the ¹H fat and water resonances of the human body. For example, at B₀=0.6 Tesla the same fluorine-containing magnetic marker material 62 (trifluoroacetic acid/water solution) suitably used for generating ¹⁹F resonance has also been found to provide a chemically shifted ¹H magnetic resonance that is sufficiently chemically shifted in frequency to enable selective excitation of the chemically shifted ¹H marker resonance without substantial excitation of the ¹H fat/water resonances.

Hence, in some embodiments the example trifluoroacetic acid solution 62 is used as the marker material at both low fields (e.g., B₀=0.23 Tesla) and high fields (e.g., B₀=0.6 Tesla). For low fields, the ¹⁹F marker resonance is excited; at high fields, the chemically shifted ¹H resonance is excited. The skilled artisan can select other marker materials that are suitably used at these or other magnetic fields. Moreover, in some contemplated embodiments, a ¹H water or ¹H fat marker resonance is excited along with the ¹H patient resonance, and the close proximity of the marker coils to the marker material in the fiducial markers 31, 32, 33 provides sufficient selectivity to distinguish the marker signals from ¹H patient resonance signals.

FIGS. 7A and 7B show example Fourier-transformed frequency domain spectra measured for “Ch0” and “Ch1”, respectively, for a selected one-dimensional projection. In FIGS. 7A and 7B, two peaks arise from the first fiducial marker 31: a peak in the “Ch0” spectrum due to the coil 70, and a peak in the “Ch1” spectrum due to the coil 84. These peaks due to the first fiducial marker 31 are labeled “#1” in FIGS. 7A and 7B. The second fiducial marker 32 contributes a peak to the “Ch0” spectrum of FIG. 7A. This second peak due to the second fiducial marker 32 is labeled “#2”. Similarly, the third fiducial marker 33 contributes a peak to the “Ch1” spectrum of FIG. 7B, which is labeled “#3”.

Although the peaks are labeled “#1”, “#2”, or “#3” in FIGS. 7A and 7B, thus identifying the peaks with specific fiducial markers for illustrative purposes, it will be appreciated that the peaks are not identified with specific fiducial markers in the as-acquired spectra. It will be appreciated that in some positions and orientations of the fiducial assembly 30, one or both of the “#1” peaks may overlap with the “#2” peak and/or the “#3” peak, or the peaks may be in a state of high spatial symmetry, or there may be other ambiguities in identifying specific peaks with specific fiducial markers.

Accordingly, the position/orientation processor 44 of FIG. 1 performs a method by which the peaks in the “Ch0” and “Ch1” spectra can be unambiguously identified with specific ones of the fiducial markers 31, 32, 33. A suitable method is described below. Once the peaks are unambiguously identified in each one-dimensional projection spectrum, the spatial location of each fiducial marker 31, 32, 33 in that projection direction can be determined based on the spatial relationship of the frequency encoding of the projection. This produces marker location information in the basis of the selected one-dimensional projections. This location information along with the a priori known relationships among the fiducials is converted to a suitable orthonormal basis to derive position and orientation information in the coordinate system of the scanner 10, in an anatomical coordinate system, or in another suitable coordinate system.

In a suitable processing method, the “Ch0” and “Ch1” spectra for each projection are stored in a complex floating point representation, and four projection directions are employed, each orthogonal to a different one of four faces of a tetrahedron. This selection of four projection directions advantageously creates an overdetermined system enabling self-consistency checks, detection of failures due to measurement errors, processing errors, or the like, and failure recovery for errors in a single projection direction.

Optionally, the acquired “Ch0” and “Ch1” spectra are apodized in the time domain, for example by setting the first and last 128 samples of a 512 sample projection data set to zero. Such apodization produces insubstantial loss of information as long as the peaks from the fiducial markers 31, 32, 33 in the projection spectra are at least several pixels wide. This optional apodization reduces the free induction decay tail of the 180° radio frequency pulse 114 (labeled in FIG. 6) and substantially boosts the signal to noise ratio.

With continuing reference to FIGS. 7A and 7B and with further reference to FIGS. 8A and 8B, the peaks due to the coils 70, 84 of the first fiducial marker 31 (that is, the peaks labeled “#1” in FIGS. 7A and 7B) are identified by taking advantage of the arrangement of the fiducial markers 31, 32, 33 in which the coils 70, 84 of the first marker 31 are orthogonal and have handedness which is opposite to that of the coils 74, 80 of the second and third markers 32, 33. The frequency domain spectra of FIGS. 7A and 7B (after optional apodization) are pointwise multiplied using a cross-product-like operation. Denoting the Fourier transform of the “Ch0” data for projection “n” as f_{ch0,n}, and denoting the Fourier transform of the “Ch1” data for projection “n” as f_{ch1,n}, the pointwise multiplicative operation is defined as:
bₙ=Re{f_{ch0,n}}·Im{f_{ch1,n}}−Re{f_{ch1,n}}·Im{f_{ch0,n}} (1),
where bₙ is the result of the pointwise multiplicative operation, and is shown in FIG. 8A. Due to the handedness property of the data, the peaks “#2” and “#3” due to the coils 74, 80 of the fiducial markers 32, 33 are small or negative, and are suitably set to zero or otherwise discarded. Thus, the result spectrum bₙ shown in FIG. 8A includes only a single peak, labeled “#1”, corresponding to the multiplicatively combined signals of the coils 70, 84 of the first fiducial marker 31.

The multiplicative spectrum bₙ is optionally processed to improve the data, for example by optional smoothing and/or Fourier interpolation. In one such optional approach, zero padding is applied symmetrically to the positive and negative frequencies of bₙ to produce a 5120 point data set, and a Fourier convolution smoothing is applied using a one-dimensional estimated projection shape of one of the fiducial markers in the frequency domain with appropriate zero padding. The result of such optional smoothing and interpolating is shown in FIG. 8B, and is analyzed by a suitable peak search algorithm to identify the location of the first fiducial marker 31 in the projection denoted “n”. This location of the first fiducial marker 31 in the projection “n” is denoted as “l_{n,1}”, and is suitably expressed as a spatial location along the projection “n” based upon the spatial frequency encoding used in acquiring the projection “n”.

It will be appreciated that the “#1” peak in the “Ch0” spectrum due to the coil 70 and the “#1” peak in the “Ch1” spectrum due to the coil 84 should occur at the same frequency since they are spatially coincident at the first fiducial marker 31. If these peaks do not overlap due to a frequency miscalibration of one of the receiver channels 40, 42 or due to another problem in the tracking system, this will generally become apparent during processing because in that case the non-overlapping “#1” peaks of “Ch0” and “Ch1” will not multiply together to provide a “#1” peak in the bₙ spectrum. Thus, a data consistency check is provided. Moreover, in the example of FIGS. 7A, 7B, 8A, and 8B, the peaks of the second and third fiducial markers 32, 33 do not overlap. Accordingly, these peaks are substantially eliminated, that is, are reduced close to zero, by the multiplicative operation of Equation (1).

With reference to FIGS. 9A, 9B, and 9C, the situation when the second and third fiducial markers 32, 33 strongly overlap is illustrated. FIGS. 9A and 9B show example Fourier-transformed frequency domain spectra measured for “Ch0” and “Ch1”, respectively, for a selected one-dimensional projection in which the peaks “#2” and “#3” due to the second and third fiducial markers 32, 33, respectively, strongly overlap. FIG. 9C shows the multiplicative product bₙ produced by Equation (1) applied to the spectra of FIGS. 9A and 9B. Due to the overlap of peaks “#2” and “#3”, the multiplicative operation of Equation (1) does not eliminate the “#2” and “#3” peaks, but rather produces a negative (i.e., different phase) peak due to their multiplicative combination. This multiplicatively combined negative peak is labeled “#2 & #3” in FIG. 9C. By discarding negative values of bₙ (for example, by setting negative values of bₙ equal to zero) the spectrum of FIG. 9C is again can be reduced to a single positive peak corresponding to the first fiducial marker 31. This positive peak is labeled “#1” in FIG. 9C. Smoothing and interpolating operations are optionally performed on the spectrum of FIG. 9C after removal of the extraneous negative peak to produce improved peak definition similar to that shown in FIG. 8B, from which the precise position of the “#1” peak can be identified.

It will be appreciated that rather than having the first fiducial marker 31 produce the positive peak in the bₙ spectrum, the coils 70, 74, 80, 84 could instead be wound such that the two coils 70, 84 of the first fiducial marker 31 produce a negative peak while the two coils 74, 80 of the second and third fiducial markers 32, 33, when spatially overlapping, produce a positive peak. This arrangement would allow identification of the first fiducial marker 31 as the negative peak of bₙ.

With the peak associated with the first fiducial marker 31 identified in the “Ch0” spectrum, the remaining peak in the “Ch0” spectrum is identified as being due to the coil 74 of the second fiducial marker 32. Similarly, with the peak associated with the first fiducial marker 31 identified in the “Ch1” spectrum, the remaining peak in the “Ch1” spectrum is identified as being due to the coil 80 of the third fiducial marker 33. One suitable method for identifying these “#2” and “#3” peaks unambiguously and with high precision (even when the “#1” peak partially or totally overlaps the “#2” or “#3” peak) employs least squares fitting in the time domain as follows.

With reference to FIGS. 10A, 10B, and 10C, a time domain approximation of the signal “#1” produced by the first fiducial marker 31 is generated. FIG. 10A shows an apodized shape of an ideal ball sized to match the first fiducial marker 31 when positioned at the center of the imaging region (that is, location=0). In one approach, the apodized shape of FIG. 10A is generated by applying an inverse Fourier transform to the one-dimensional estimated frequency domain fiducial marker projection shape used in the convolutional smoothing discussed with reference to FIG. 8B. To account for the generally non-zero spatial location “l_{n,1}” of the first fiducial marker 31 in the projection “n”, the Fourier shift theorem is applied. The Fourier shifting function in the time domain is given by:
f_{shift}=exp[i·(m−N/2)·π·l_{n,1}] (2),
where i is the imaginary unit, N is the number of sample data points, and m indexes the sample data points in the time domain. FIG. 10B depicts the shifting function f_{shift} in the time domain for a slightly off-center position. The time domain product of the approximation of the fiducial marker (FIG. 10A) and the shifting function (FIG. 10B) is depicted in FIG. 10C, and approximates the time domain signal of the first fiducial marker 31 at location l_{n,1} in projection “n”.

The time-shifted shape of FIG. 10C is separately fitted to the “Ch0” and “Ch1” data after all projections (for example, all four tetrahedral projection directions) have been acquired. For every projection direction “n”, a complex least squares fit of the time-shifted shape of FIG. 10C is performed to the time domain “Ch0” and “Ch1” data separately, yielding two sets of four coefficients a_{ch0,n}, and a_{ch1,n}. These are averaged over the eligible values, excluding cases where the projections of the two fiducial markers of the receiver channel overlap, to give scaling coefficients a_{ch0} and a_{ch1}. The time-shifted shape of peak “#1” (approximated for example in FIG. 10C for one specific location l_{n,1}) for each projection “n” is multiplied by the common coefficients a_{ch0} and a_{cb1} and subtracted from the corresponding time domain “Ch0” and “Ch1” data for that projection “n” to yield time domain data containing only the “#2” peak (for “Ch0”) or the “#3” peak (for “Ch1”).

This process is illustrated in FIGS. 11A, 11B, and 11C for the “Ch0” data and a specific projection “n”. FIG. 11A shows the time-shifted shape of FIG. 10C multiplied by the averaged complex least squares scaling fitting coefficient a_{ch0} (smooth line) and the measured time domain “Ch0” data (noisy line). FIG. 11B shows the residual produced by subtracting the smooth line of FIG. 11A (the time shifted shape of FIG. 10C scaled by fitted coefficient a_{ch0}) from the noisy line of FIG. 11A (the “Ch0” time domain data). FIG. 11C shows the amplitude spectrum of the Fourier transform of the data of FIG. 11B. In FIG. 11C, the dashed peak represents the “#1” peak which was substantially removed by the processing of FIGS. 11A and 11B. The Fourier spectrum of FIG. 11C (with the “#1” peak removed) is suitably processed by a peak search algorithm to identify the location of the second fiducial marker 32 in the projection denoted “n”, which is suitably denoted “l_{n,2}”. Similar processing is applied to the “Ch1” data to identify the location of the third fiducial marker 33 in the projection denoted “n”, which is suitably denoted “l_{n,3}”.

Rather than removing the “#1” peak by subtraction, that peak could be accounted for in other ways. For example, a least squares fit of both the “#1” and “#2” peaks (for “Ch0”) could be performed simultaneously, allowing the position of the “#2” peak to be a fitted parameter. In this approach the “#1” peak is not removed, but is accounted for in the fitting process.

The locations “l_{n,k}”, where “n” denotes the projection (having values n=1, 2, 3, 4 for the four directions in the example tetrahedral projection direction configuration) and “k” denotes the fiducial marker (having values k=1, 2, 3 for first, second, and third fiducial markers 31, 32, 33, respectively) are converted to a selected orthonormal basis (such as the coordinate system of the scanner 10, or an anatomical coordinate system associated with a human imaging subject) as follows. For each fiducial marker “k”, a location vector Iₖ=(lₙ)ₖ is defined. For four projection directions (n=1, 2, 3, 4), each location vector Iₖ is a 4×1 vector, and there are three such vectors corresponding to the three fiducial markers 31, 32, 33 indexed k=1, 2, 3. To convert to the selected orthonormal basis, the overdetermined system Acₖ=Iₖ is solved for cₖ, where A is a 4×3 matrix containing the projection directions expressed in the desired orthonormal basis and cₖ is a 3×1 vector specifying the position of fiducial marker “k” in the desired orthonormal basis. This overdetermined system can be suitably solved by least squares fitting or another method. Optionally, information about the accuracy and precision of the previous processing can be incorporated into the least squares fitting by multiplying both sides of the equation Acₖ=Iₖ from the right with a diagonal weight matrix.

From the positions of fiducial markers given by cₖ where k=1, 2, 3 for fiducial markers 31, 32, 33, respectively, a rotation matrix can be constructed by defining, for example: a=c₁c₂; b=c₁-c₃; d=−a-b; e=a×b; and f=e×d. A fully qualified, orthonormal rotation matrix can be written as R={|e|, |f|, |d|}, where the vertical bars “|·|” denote normalization. By choosing the generally least noisy coordinate to represent the translation of the fiducial assembly 30, the augmented rotation matrix can be written as:

$$T = ( \begin{matrix} & R & & {c}_{1} \\ 0 & 0 & 0 & 1\end{matrix} ) , \qquad ( 3 )$$

where for illustrative purposes the coordinate c₁ is selected as the least noisy coordinate for representing the translation of the fiducial assembly 30.

The described approach advantageously enables tracking consistency checking. In one approach, the fitting residual of the equation Acₖ=Iₖ for each fiducial is examined for consistency. In another approach, the fiducial location vectors (known from calibration) of a non-rotated probe in the origin are multiplied with the calculated matrix T. Summing up distances between fiducial centers calculated this way and the ones from the coordinate transformation provides a consistency check for T that also takes into account the known shape and dimensions of the probe.

With reference to FIGS. 12A, 12B, and 12C, the position and orientation of the fiducial assembly 30 was measured using the above techniques, with the fiducial assembly 30 mounted in a goniometric jig that provided precise control of the Euler ZYZ angles (also known as the Euler Y-convention), in which the first rotation φ is around the z-axis, the second rotation θ is around the y′-axis, and the third rotation ψ is around the doubly rotated z″ axis. For determination of angular noise and rotation-dependent systematic errors, a set of measurements with fixed θ and ψ and varying φ were performed. The first fiducial marker 31 was positions approximately at the isocenter of the region of interest 12 of the magnetic resonance imaging scanner 10. Position/orientation measurements were performed (100 measurements acquired over 10 seconds) during which time the angle φ was varied through a 90° interval. Of the two fixed angles θ and ψ, the angle θ was chosen as the measurand, since the non-uniqueness of Euler angles (in contrast to the rotation matrix) substantially mixes the values of ψ and φ together at low values of angle θ. The measured variance of angle θ was divided into: (i) a low frequency (lowest 2% of frequencies) component, which was taken to represent the systematic errors of the algorithm; and (ii) a high frequency component interpreted as statistical fluctuation. The results are presented in FIGS. 12A and 12C. FIG. 12A shows the measured standard deviation of θ, where the data is regridded, measurement points lie at θ=10°, 20°, . . . 70°, and ψ=15°, 30°, 45°, 52°, 60°, 67°, 75°, and 82°. FIG. 12C shows the measured rotation-dependent errors of θ. For comparison, theoretical predictions of the standard deviation assuming linear dependence between inverse signal-to-noise ratios of the derived channels representing each peak “#1”, “#2”, “#3” and statistical angle fluctuation, are plotted in FIG. 12B. The deviations or errors shown in FIG. 12A compare favorably with the theoretical prediction of FIG. 12B.

Positional noise was investigated by selecting angle combinations, which produce differing signal-to-noise ratios for the derived channel b, representing peak “#1” of the first fiducial marker 31, and taking measurement runs with the fiducial assembly 30 kept stationary. The results showed a positional noise having a standard deviation of 0.17 millimeters (with all coils perpendicular to the static B₀ magnetic field) to 0.35 millimeters (limit of algorithmic stability). These results comport with the angular noise figures, indicating that translational movement does not affect accuracy.

There is a limit to the tracking speed of the fiducial assembly 30. When one of the fiducial markers 31, 32, 33 moves in the direction of the applied gradient during echo time, phase errors result. Experiments have indicated that such phase errors are tolerable at least for speeds of up to about 40 millimeters/second. The fiducial assembly 30 should be located within the homogeneous volume of the scanner 10. For maximum accuracy, the coil normals 72, 76, 82, 86 should have angles larger than about 20° respective to the direction of the static B₀ magnetic field. With brief reference back to FIG. 2, it will be appreciated that this latter condition can generally be met by judicious selection of the mounting orientation of the fiducial assembly 30 on the interventional instrument 20.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

1. A magnetic resonance position and orientation marking system comprising:
a fiducial assembly including at least three fiducial markers each coupled with at least one magnetic resonance receive coil, at least one of the fiducial markers having at least one of: (i) marker nuclei selectively excitable over ¹H fat and water resonance, and (ii) a plurality of magnetic resonance receive coils; and
at least two magnetic resonance receive channels receiving magnetic resonance signals from the at least three fiducial markers responsive to excitation of magnetic resonance in said at least three fiducial markers by an associated magnetic resonance imaging scanner.

2. The system as set forth in claim 1, wherein the at least two magnetic resonance receive channels includes:
a first magnetic resonance receive channel connected with (i) a first coil having a first spatial orientation and coupled with a first one of the at least three fiducial markers, and (ii) a second coil having a second spatial orientation different from the first orientation and coupled with a second one of the at least three fiducial markers; and
a second magnetic resonance receive channel connected with (i) a third coil having the first spatial orientation with opposite polarity respective to the first coil and coupled with a third one of the at least three fiducial markers, and (ii) a fourth coil having the second spatial orientation with opposite polarity respective to the second coil and coupled with the first one of the at least three fiducial markers.

3. The system as set forth in claim 1, wherein the at least one magnetic resonance receive coil coupled with each of the at least three fiducial markers include:
at least two receive coils having spatial orientations different from one another and coupled with a first one of the at least three fiducial markers.

4. The system as set forth in claim 1, wherein the at least two magnetic resonance receive channels include:
a first magnetic resonance receive channel connected with a series combination of: (i) a first coil having a first spatial orientation and coupled with the first one of the at least three fiducial markers, and (ii) a second coil having a second spatial orientation different from the first orientation and coupled with a second one of the at least three fiducial markers; and
a second magnetic resonance receive channel connected with a series combination of: (i) a third coil having the first spatial orientation with opposite polarity respective to the first coil and coupled with a third one of the at least three fiducial markers, and (ii) a fourth coil having the second spatial orientation with opposite polarity respective to the second coil and coupled with the first one of the at least three fiducial markers.

5. The system as set forth in claim 4, wherein the first and second spatial orientations are mutually orthogonal.

6. The system as set forth in claim 4, wherein a plurality of one-dimensional projection excitations excite a plurality of one-dimensional projections, the system further including:
a processor causing a magnetic resonance method to be performed that determines a position and orientation of the fiducial assembly, the method including:
collecting magnetic resonance signals received by the first and second magnetic resonance receive channels for the plurality of one-dimensional projections produced by the associated magnetic resonance imaging scanner,
for each projection, distinguishing magnetic resonance signals of the first and fourth coils from magnetic resonance signals of the second and third coils based on phase of the magnetic resonance signals,
for each projection, determining a location of the first one of the at least three fiducial markers along the projection based on the magnetic resonance signals of at least one of the first and fourth coils,
for each projection, determining locations of the second and third of the at least three fiducial markers along the projection based on the magnetic resonance signals of the second and third coils respectively, and
determining the position and orientation of the fiducial assembly based on the determined locations of the first, second, and third of the at least three fiducial markers along each of the plurality of projections.

7. The system as set forth in claim 6, wherein the plurality of one-dimensional projections lie along four different directions each orthogonal to a different one of four faces of a tetrahedron.

8. The system as set forth in claim 7, wherein the determining of a position and orientation of the fiducial assembly based on the determined locations of the first, second, and third of the at least three fiducial markers along each of the plurality of projections includes:
constructing an augmented rotation matrix in a selected coordinate system from the determined locations of the first, second, and third of the at least three fiducial markers.

9. The system as set forth in claim 6, wherein the distinguishing of magnetic resonance signals of the first and fourth coils from magnetic resonance signals of the second and third coils includes:
for each projection, Fourier transforming the magnetic resonance signals received by the first and second magnetic resonance receive channels; and
for each projection, multiplying together the Fourier transformed magnetic resonance signals received by the first and second magnetic resonance receive channels, the multiplying selected to produce a sign reversal of one of: (i) magnetic resonance signals of the first and fourth coils, and (ii) magnetic resonance signals of the second and third coils.

10. The system as set forth in claim 6, wherein the distinguishing of magnetic resonance signals of the first and fourth coils from magnetic resonance signals of the second and third coils includes:
for each projection, Fourier transforming the magnetic resonance signals received by the first and second magnetic resonance receive channels; and
for each projection, multiplying together the Fourier transformed magnetic resonance signals received by the first and second magnetic resonance receive channels the multiplying eliminating non-overlapping magnetic resonance signals of the second and third coils.

11. The system as set forth in claim 6, wherein the distinguishing of magnetic resonance signals of the first and fourth coils from magnetic resonance signals of the second and third coils includes:
approximating a time-domain shape of the magnetic resonance signal of the first and fourth coils;
for each projection, temporally shifting the approximated time-domain shape based on the determined location of the first one of the at least three fiducial markers along the projection; and
for each projection, determining the magnetic resonance signals of the second and third coils by mathematically accounting for or removing the approximated and temporally shifted time-domain shape of the magnetic resonance signal of the first and fourth coils.

12. The system as set forth in claim 1, wherein each of the at least three fiducial markers include fluorine marker nuclei, and the at least two magnetic resonance receive channels are tuned to a magnetic resonance frequency of fluorine nuclei.

13. The system as set forth in claim 12, wherein the at least two magnetic resonance receive channels are tuned to the ¹⁹F magnetic resonance frequency.

14. The system as set forth in claim 1, wherein each of the at least three fiducial markers includes chemically shifted ¹H marker nuclei having a chemical frequency shift enabling selective excitation of the chemically shifted ¹H marker nuclei over ¹H fat and water resonance, the at least two magnetic resonance receive channels being tuned to the resonance frequency of the chemically shifted ¹H marker nuclei.

15. The system as set forth in claim 1, wherein each of the at least three fiducial markers includes a trifluoroacetic acid solution including at least trifluoroacetic acid and water, and the at least two magnetic resonance receive channels are tuned to one of: (i) a magnetic resonance frequency of fluorine nuclei, and (ii) a magnetic resonance frequency of chemically shifted ¹H nuclei of the trifluoroacetic acid solution.

16. The system as set forth in claim 15, wherein the trifluoroacetic acid solution further includes a T2 relaxation time shortening agent.

17. A method for determining position and orientation of a fiducial assembly including at least three fiducial markers, the method comprising:
exciting magnetic resonance in the at least three fiducial markers, each fiducial marker being coupled with at least one magnetic resonance receive coil, at least one of the fiducial markers having at least one of: (i) marker nuclei selectively excitable over ¹H fat and water resonance, and (ii) a plurality of magnetic resonance receive coils; and
receiving magnetic resonance signals from the excited at least three fiducial markers via at least two magnetic resonance receive channels.

18. The method as set forth in claim 17, wherein the exciting and receiving is performed along a plurality of projection directions, the method further comprising:
determining locations of each of the at least three fiducial markers along each projection based on the received magnetic resonance signals; and
determining the position and orientation of the fiducial assembly based on the determined locations of the at least three fiducial markers.

19. The method as set forth in claim 18, wherein the receiving of the magnetic resonance signals includes:
receiving via the first magnetic resonance signal channel an additive combination of: (i) a first resonance signal component from a first coil coupled with the first one of the at least three fiducial markers and having a first polarization direction and (ii) a second resonance signal component from a second coil coupled with a second one of the at least three fiducial markers and having a second polarization direction different from the first polarization direction; and
receiving via the second magnetic resonance signal channel an additive combination of: (i) a third resonance signal component from a third coil coupled with a third one of the at least three fiducial markers and having the first polarization direction with opposite polarity respective to the first coil and (ii) a fourth resonance signal component from a fourth coil coupled with the first one of the at least three fiducial markers and having the second polarization direction with opposite polarity respective to the second coil.

20. The method as set forth in claim 19, wherein the determining of locations of each of the at least three fiducial markers along each projection based on the received magnetic resonance signals includes:
for each projection, separating first and fourth resonance signal components from the second and third resonance signal components based on the phases;
for each projection, determining a location of the first one of the at least three fiducial markers based on the first and fourth resonance signal components; and
for each projection, determining locations of the second and third of the at least three fiducial markers based on the second and third resonance signal components.

21. The method as set forth in claim 17, wherein the exciting and receiving includes:
exciting and receiving ¹⁹F magnetic resonance signals from each of the at least three fiducial markers.

22. The method as set forth in claim 17, wherein the exciting and receiving includes:
exciting and receiving ¹H marker magnetic resonance signals from each of the at least three fiducial markers, the I¹H marker magnetic resonance signals being chemically shifted from ¹H fat and water magnetic resonances enabling selective excitation of the ¹H marker magnetic resonance signals over ¹H fat and water magnetic resonances.

23. A computing apparatus programmed to perform the method of claim 17.
